(19) United States
(12) Patent Application Publication
(10) Pub. No.: US 2009/0183733 A1
(43) Pub. Date: Jul. 23, 2009

(54) Nebulizer delivery device and system for animals and method of using same

(76) Inventors: Everette Allen Deaver, JR., Winter Park, FL (US); Christopher Dold, Winter Garden, FL (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/015,749

(22) Filed: Jan. 17, 2008

(51) Int. Cl. A61M 11/00

(52) U.S. Cl. 128/200.21

(57) ABSTRACT

A nebulizer delivery device for administering a nebulized drug to an animal having a means for connecting the device to a drug source, a reservoir for holding the nebulized drug in communication with the connecting means, at least one air inlet valve and at least one inhalation one-way valve for delivering the drug to the animal, central chamber also in communication with the reservoir and at least one exhalation one-way valve, and a mask for receiving the nebulized drug and for fitting over a breathing portion of the animal. A nebulizer system and method of administering a nebulized drug to an animal via the system are also described and claimed.

### FIELD OF THE INVENTION

The present invention relates generally to nebulizers and, in particular, to a novel nebulizer delivery device and system for treating animals and a method of using same.

### BACKGROUND OF THE INVENTION

Nebulizers are widely used in the medical field for respiratory therapy and, in particular, delivering medication or drugs to patients' lungs through inhalation. Nebulizers are typically designed to break down liquid or water-soluble medication into small particles resembling a mist. Atomization of the aerosol mist typically occurs by feeding pressurized air into a nebulizer device. The patient typically inhales through an outlet orifice located at the side or top of the nebulizer allowing the medicated mist to enter its respiratory system, including its lungs and airways, thereby resulting in the administration of the medication to the patient. Medical nebulizers that nebulize a medicated fluid into an aerosol for inhalation by a patient are well-known devices commonly used for the treatment of various conditions and diseases. Typically, nebulizers utilize a diverter, baffle or infringer to direct a gas across a liquid channel to create a venturi effect causing the liquid channel to be entrained into the gas stream. As a result of the nebulizing process, the fluid is transformed into an aerosol, that is, the fluid is caused to form small particles in a range suitable for the intended therapy, such as inhalation therapy.

Important considerations in the design of a nebulizer are the timing and dosage regulation of the aerosolized fluid. In some nebulizer designs, a continuous stream of pressurized gas entrains the fluid against the diverter to constantly generate an aerosol until the fluid in a reservoir is depleted. Effective and economical nebulizer therapy includes the ability to quickly generate an aerosol within a desired particle size range. An effective nebulizer preferably provides these features synchronously with the inhalation of the patient. It is also desirable that a nebulizer has adequate sensitivity to quickly respond to a patient's inhalation while not adversely restricting the inhalation.

Treatment of humans with nebulizers takes usually about 2-5 minutes to complete but it can take several hours until the desired result is achieved. If the medication does not achieve the desired result, the dose of medication may be increased or a different medication may be used. Nebulized medications are usually water-soluble and depending on the type and severity of condition to be treated, nebulized medications can include, for example, steroids, demucelents, bronchodilators and antibiotics.

To date, sporadic attempts have been made to nebulize various animals with less than favorable results. Attempts using a metered dose inhaler or MDI have been the most common. There are several reasons for the shortcomings in treating animals using nebulizers including primarily that the design of nebulizers used on animals have been typically adapted from nebulizers originally used by humans. Most nebulizers for humans require human input including especially that the human patient first exhale before placing the nebulizer device on, over or in his/her mouth and then manually form a seal between the nebulizer and the mouth region before activating the nebulizer upon inhalation. Unlike humans, it is difficult and often impossible for animals to provide similar input in order to effectively treat them with a nebulized drug.

For example, marine mammals breathe through a blow hole and exchange air very quickly before they dive below the surface of water. It is estimated that an adult bottle nosed dolphin exhales and inhales approximately 5 liters of air in less than one second. Because a dolphin's exhale/inhale cycle is completed so quickly, it is impossible to place a typical nebulizer used by humans over a dolphin's blow hole at precisely the time after the dolphin exhales but before it takes its next breath. Not only do the exhale/inhale cycles happen quickly, they often vary based on the position, health, age and even mood of the animal. In fact, filling a tube with a nebulized drug using an MDI and then trying to position it over the blow hole of a dolphin at just the correct time for inhalation was found to produce no clinically significant results as it was determined that most of the nebulized air was blown out of the tube before it could be inhaled.

Accordingly, a need exists for a nebulizer delivery device as well as a nebulizer system and method that effectively and efficiently delivers a nebulized drug to animals.

### SUMMARY OF THE INVENTION

It is, therefore, an object of the present invention to provide a lightweight nebulizer delivery device that effectively and efficiently administers a nebulized drug to animals.

Another object of the present invention is to provide a nebulizer system that can easily be used on animals without removing them from their natural environment.

It is a further object of the present invention to provide a nebulizer delivery device and system which enables animals to be easily treated daily or multiple times a day without undue stress or the need for extensive resources.

It is still a further object of the present invention to provide a nebulizer delivery device and system which is capable of effectively and efficiently administering more than one nebulized drug to an animal.

It is yet another object of the present invention to provide a nebulizer delivery device and system that can be used on animals having varying levels of training.

It is yet another object of the present invention to provide a method of using a nebulizer system that effectively and efficiently delivers a nebulized drug to an animal.

These and other objects of the present invention are achieved by a nebulizer delivery device having a means for connecting a drug or medication source to the device. The nebulizer delivery also includes a reservoir that holds the nebulized drug in communication with the connecting means, at least one air inlet valve, and at least one inhalation one-way valve for delivering the nebulized drug to the animal. A central chamber is in communication with the reservoir, at least one exhalation one-way valve, and a mask for receiving the nebulized drug and for fitting over a breathing portion of the animal.

The objects of the present invention are also achieved by a nebulizer system which includes at least one source of a drug for treating the animal that is detachably connectable to a nebulizer delivery device as described further herein a nebulized drug to an animal via the system are also described and claimed.

The invention is also achieved by a novel method of administering a drug via a nebulizer system to an animal. More specifically, the method includes connecting at least one source of a drug to a nebulizer device having a connecting means, a reservoir, at least one air inlet valve, inhalation and exhalation one-way valves, a central chamber and a mask. As further described below, the mask is placed over a breathing portion of the animal and the source is activated so that the drug fills the reservoir. The mask is held in place until the animal completes at least one exhalation/inhalation cycle during which at least a portion of the nebulized drug is administered to the animal.

### BRIEF DESCRIPTION OF SEVERAL VIEWS OF THE DRAWINGS

Reference will now be made to the drawings, wherein like parts are designated by like numerals, and wherein

FIG. 1 is a top plan view of an embodiment of the nebulizer system of the present invention;

FIG. 2 is a vertical sectional view taken on line 2-2 of FIG. 1 showing flow upon inhalation by an animal;

FIG. 3 is a vertical sectional view taken on line 2-2 of FIG. 1 showing flow upon exhalation by an animal;

FIG. 4 is a vertical sectional view taken on line 4-4 of FIG. 2;

FIG. 5 is a vertical sectional view taken on line 5-5 of FIG. 3; and

FIG. 6 is a side respective view showing another embodiment of the nebulizer system of the present invention used on a horse.

### DETAILED DESCRIPTION OF THE INVENTION

Certain terminology may be used in the following description for convenience only and is not limiting. The words “right,” “left,” “lower,” and “upper” designate directions in the drawings to which reference is made. The terminology includes the words above specifically mentioned, derivatives thereof and words of similar import.

The present invention overcomes the shortcomings of known nebulizers, nebulizer systems and methods of using same by providing a versatile nebulizer delivery device and system for nebulizing a drug and administering the nebulized drug to an animal with a medical condition in order to effectively treat the animal.

The term “drug” as used herein means any drug capable of being nebulized regardless of whether it is physiologically active and includes, but is not limited to, known medications used to treat animals as well as medications to be discovered in the future. Such medications include water-soluble medications including steroids, bronchodilators, demucelents and antibiotics.

The term “animal” as used herein means any animal capable of being nebulized and includes, but is not limited to, dogs, horses, zebras, lions, and tigers as well as marine mammals, including dolphins and whales.

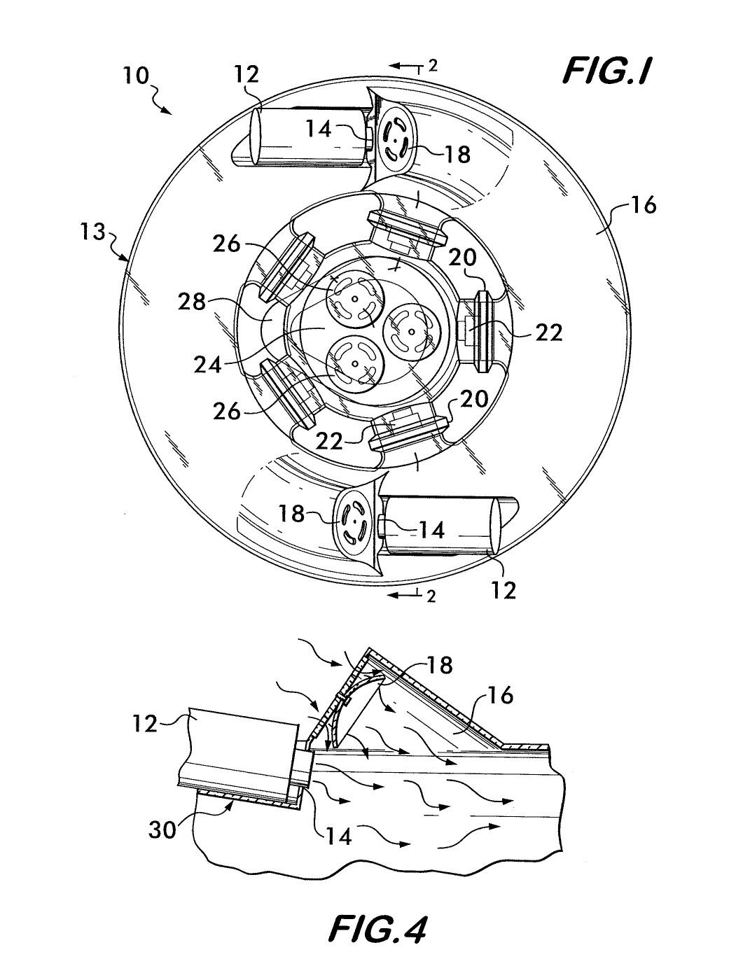

Referring in more detail to the application drawings, wherein like parts are indicated by like reference numerals, and initially to the FIGS. 1-5 embodiment, the system constructed according to the present invention is generally indicated as numeral 10. The nebulizer system 10 generally includes the combination of: 1) at least one source 12 of a drug for nebulization and; 2) a nebulizer delivery device 13. The source 12 of a drug for nebulization includes, but is not limited to, known nebulizers including, preferably, jet nebulizers as they produce a positive pressure and a swirling effect in the nebulizer delivery device (described below) thereby reducing condensation of the nebulized drug while it remains in the delivery device. Jet nebulizers capable of producing flow rates of at least 4 liters/minute are most preferred. For example, the MicroAir Nebulizer by Omron Healthcare, Inc. (Bannockburn, Ill.) has proven to be effective.

In order to be effective, the source 12 must be capable of effectively and efficiently breaking down liquid or water-soluble drugs into small particles resembling an aerosol mist.

The source 12 must also be capable of delivering the nebulized drug to the nebulizer delivery device 13 such that an adequate flow rate of the nebulized drug is maintained in the delivery device 13 in order to minimize condensation of the drug until such time as the animal inhales the nebulized drug.

Preferably, the source 12 is self-contained meaning that it contains the drug to be nebulized under pressure in a vile, canister or other similar object such that the source 12 itself is what is commonly known as a nebulizer. However, other types of sources can be used provided they are capable of delivering either directly or in combination with at least one other apparatus, device, object or input, a nebulized drug at a sufficient flow rate and pressure to the nebulizer delivery device 13 such that condensation of the nebulized drug is minimal. For example, the source 12 containing the drug to be nebulized can be in communication with a hose and a pneumatic pump such that pressurization of the drug occurs in a separate step prior to nebulizing the drug and delivering it to the delivery device 13.

It is also preferred, as shown in FIG. 1, that the source 12 be directly connected to the nebulizer delivery device 13 such that the source 12 and the nebulizer device 13 are in close proximity as this will serve to help reduce the likelihood that the drug will condense inside the delivery device 13. However, the source 12 can be in a remote location as long as it is delivered to the nebulizer device 13 such that a significant amount of condensation of the drug does not occur inside the delivery device 13.

It has been found that MDI's are not preferred sources because the volume of nebulized air needed to treat an animal using known MDI's is very difficult and expensive to produce. Also, several MDI canisters are required for each treatment procedure and synchronizing the release of the drug from the MDI upon inhalation by the animal is extremely difficult. This, however, is not intended to limit the possible future use of MDI's with various embodiments of nebulizer delivery device 13 of the present invention as advances and/or improvements are made to MDI's. Additionally, to date, MDI's are only available for human use and therefore only contain drugs used by humans. In contrast, jet nebulizers are capable of producing a sufficient flow rate of the nebulized drug and a sufficient volume of nebulized drug to the nebulizer delivery device 13 between breaths of an exhalation/inhalation cycle of many different animals.

The number of sources 12 required will depend on the amount of drug contained at or in the source 12 as well as type of animal being treated since the size of animals' respiratory systems vary as do their breathing patterns. FIG. 1 shows two sources 12 in communication with the nebulizer delivery device 13 such that the sources 12 are connected to connecting means 14. The source 12 should be capable of delivering a sufficient quantity of drug to effectively treat the animal during a medical procedure. If a higher dose is required, this can be achieved by changing the concentration and/or volume of drug placed in the source(s) 12 to better address the needs of the animal.

While the system 10 of the present invention is capable of working effectively when the nebulizer delivery device 13 is in communication with a single source 12, it is preferred (as shown in FIG. 1) that the nebulized drug enters the delivery device 13 at more than one location as this will help maintain a sufficient flow of the nebulized drug in the nebulizer device 13 such that condensation is minimized. Also, the use of more than one source 12 helps ensure that an adequate volume of nebulized air is available for the animal upon inhalation. It also enables an operator (e.g., trainer, veterinarian) to replace one source 12 while another source 12 continues to supply a nebulized drug to the nebulizer delivery device 13. A further advantage of using more than one source 12 is that two or more different drugs can be administered to the animal either simultaneously or in sequence.

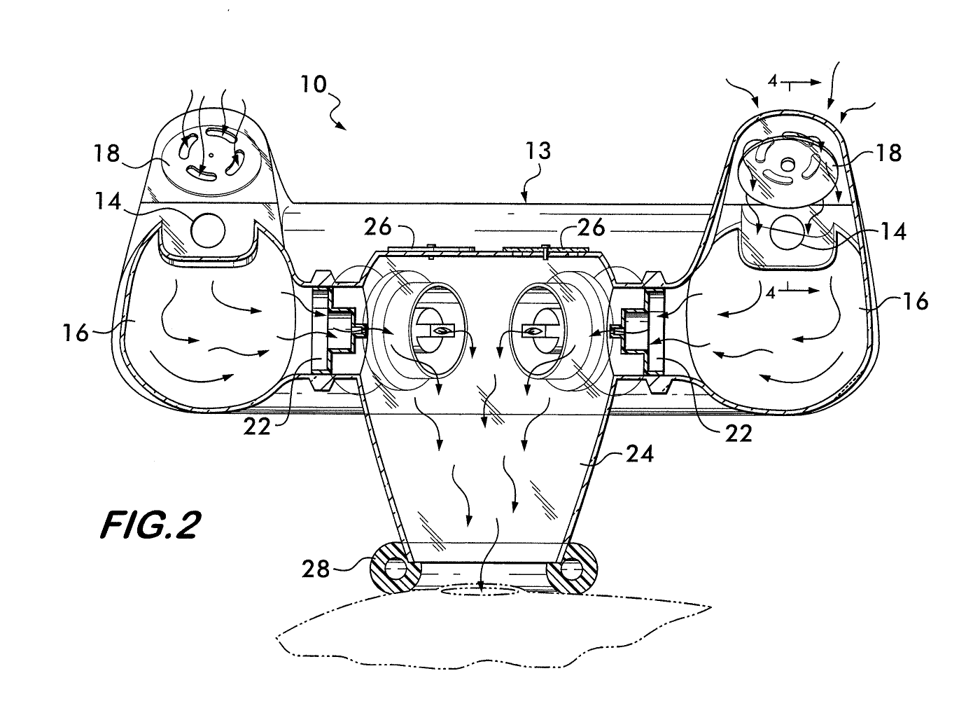

Referring to FIGS. 1 and 2, the source 12 must be connectable, either directly or indirectly, to the nebulizer delivery device 13 and, preferably, is detachably connectable such that drug sources 12 can be exchanged as necessary or desired. As shown in FIG. 4, the source 12 and nebulizer delivery device 13 are in communication thereby allowing the drug to travel from the source to the nebulizer device. As such, the nebulizer device 13 utilizes the connecting means 14 to connect, either directly or indirectly, the source 12 to the nebulizer device 13. The connecting means 14 can be any means and will depend upon whether the source 12 is connected directly or indirectly to the nebulizer device 13. If the source 12 is directly connected to the device 13, then the device 13 can be designed based on the size and shape of the source's outlet such that the source 12 easily connects to the device 13. For example, most types of male/female connectors, as well as grommets, clamps, etc., can be used effectively as connecting means. Obviously, if the source 12 is not directly connected to the nebulizer device 13, the connecting means 14 will need to accommodate, for example, the apparatus, device or input that directly connects to the nebulizer device 13. Those of ordinary skill in the art will appreciate that various types of connecting means 14 can be used effectively within the scope and spirit of the invention.

Referring again to FIGS. 1 and 2, and turning more specifically to the nebulizer delivery device 13, a reservoir 16 for holding the nebulized drug is in communication with the connecting means 14 such that the drug is deliverable from the source 12 to the reservoir 16. The location of the connecting means 14 on the reservoir 16 is an important consideration when more than one connecting means 14 is used for the nebulizer device 13. The connecting means 14 should be located such that flow in the reservoir 16 will be facilitated and condensation of the nebulized drug will be minimal. The connecting means 14 should also be positioned such that they are easily accessible.

Still referring to FIGS. 1 and 2, in addition to the connecting means 14, at least one air inlet valve 18 is in communication with the reservoir 16 such that, upon inhalation by the animal, the inlet valve 18 opens and outside air enters the reservoir 16 along with the nebulized drug. The number of air inlet valves 18 will depend on the size of the nebulizer delivery device 13 as well as the size of the animals. Those skilled in the art will appreciate that, if desired, one or more of the air inlet valves 18 (as is the case with the other valves described herein) can be covered or maintained closed such that the valve 18 does not operate (e.g., does not allow outside air to enter the reservoir 16). This may be necessary, for example, when the nebulizer delivery device 13 is too large for the animal being treated.

The reservoir 16 can be of various shapes and sizes; however, the shape is an important feature of the present invention. The reservoir 16 is designed to minimize condensation of the nebulized drug as it flows in the reservoir 16 prior to being inhaled by the animal. As such, preferably, the reservoir 16 has smooth interior walls and any joints have large radius curves to allow the most efficient flow of the nebulized drug with the least amount of condensation. Preferably, the reservoir 16 is tubular and, most preferably, donut shaped.

Although not considered to be a requirement of the present invention, other means for preventing condensation of the nebulized drug should be taken into account including, for example, using non-conductive materials and/or insulating the reservoir 16 from surrounding environmental conditions. Placement of the reservoir 16 in relation to other parts of the nebulizer device 13 should also be considered in seeking ways to minimize the formation of condensation on the interior walls of the reservoir 16. For example, if the nebulizer system 10 is used on a marine mammal, the reservoir 16 should be positioned as high above the water as possible.

The size and, in particular, the volume of the reservoir 16 should be about the same volume as the respiratory system (lungs and airways) of the treated animal. The reservoir 16 of the present invention can be adapted such that its volume can be increased or decreased as necessary. For example, the volume of the reservoir 16 can be decreased by filling a portion of the reservoir with a filling material. Also, the volume of the reservoir 16 can be increased, for example, by attaching a second reservoir (not shown) to the first reservoir 16 such that the reservoirs are in communication. Those of ordinary skill in the art will appreciate that various attaching means can be used as long as the reservoirs are in communication. For example, a tube or pipe (not shown) could join the two reservoirs after a coupling or port opening (also not shown) is installed on the walls of the two reservoirs.

The size of the reservoir 16 will depend, in part, on the size of the animal being treated. For example, since a whale's respiratory system is significantly larger than a dolphin's respiratory system, a larger reservoir 16 is necessary to treat a whale compared to that used to treat a dolphin. More particularly, whereas the respiratory system volume of a dolphin is believed to be approximately 5 liters, a respiratory system volume of a whale may be about 50 liters. As such, the reservoir 16 for treating the whale will need to be approximately ten times larger than the reservoir 16 for treating the dolphin. It is also likely that more than one source 12 of a drug is necessary to treat a large animal such as a whale. To accommodate this need for a larger reservoir 16, two or more reservoirs 16 can be placed in communication with each other by connecting them, either directly or indirectly, to each other.

Preferably, the volume of the reservoir 16 used to treat the animals will be slightly less than the respiratory system volume of the animal being treated in order to allow some air to be drawn into the reservoir 16 through the air inlet valves 18 so that essentially all of the nebulized drug is removed from the reservoir 16 prior to the next exhalation/inhalation cycle. This allows a sweeping effect to take place in the reservoir 16 which helps minimize condensation of the nebulized drug on the reservoir 16 walls.

Referring now to FIG. 2, in addition to being in communication with the connecting means 14 for receiving a nebulized drug, the reservoir 16 is also in communication with at least one exit port 20 for delivering the drug. More specifically, at least one inhalation one-way valve 22 is located in close proximity to the exit port(s) 20 and is also in communication with the reservoir 16 in order to deliver the nebulized drug from the reservoir 16 toward the animal's respiratory system. Upon inhalation by the animal, the inhalation one-way valve(s) 22 opens allowing the nebulized drug to flow out of the reservoir 16. When the animal finishes inhaling the inhalation one-way valve(s) 22 closes and prevents air from entering the reservoir 16 when the animal exhales.

The higher the resistance of the nebulizer system 10, the more difficult it will be to treat the animal. Therefore, another important feature of the nebulizer delivery device 13 is that all valves be quick-acting so that the animal experiences minimal resistance upon inhaling and exhaling. Accordingly, all valves must open and close promptly upon sensing a minimum pressure change and allow near instantaneous high flow rates. The sizes of the various valves can vary; however, preferably, the valves are larger than one-inch in diameter, most preferably about two-inches. Larger valves generally provide a quicker response time when the animal inhales and exhales such that resistance in the delivery device 13 is minimal and flow rates through the valves are adequate.

Preferably, the inhalation one-way valve 22 is a duckbill-type valve. Also, preferably, the air inlet valve 18 and an exhalation one-way valve 26 (described below) are umbrella-type valves. Both the duckbill-type and umbrella-type valves are available from Unomedical, Inc. (McAllen, Tex.). It has been found that properly sized duckbill valves do not cause the nebulized drug to condense because the direction of the nebulized drug/air mixture does not change as it passes through the inhalation one-way valves 22. The size and quantity of inhalation one-way valves 22 that need to be used will depend on the type of animal being treated such that an animal with a large respiratory system will, as discussed, require a larger reservoir 16 and therefore, preferably, utilize more than one inhalation one-way valve 22. It is preferred that more than one inhalation one-way valve 22 be used as this helps minimize the resistance in the nebulizer system.

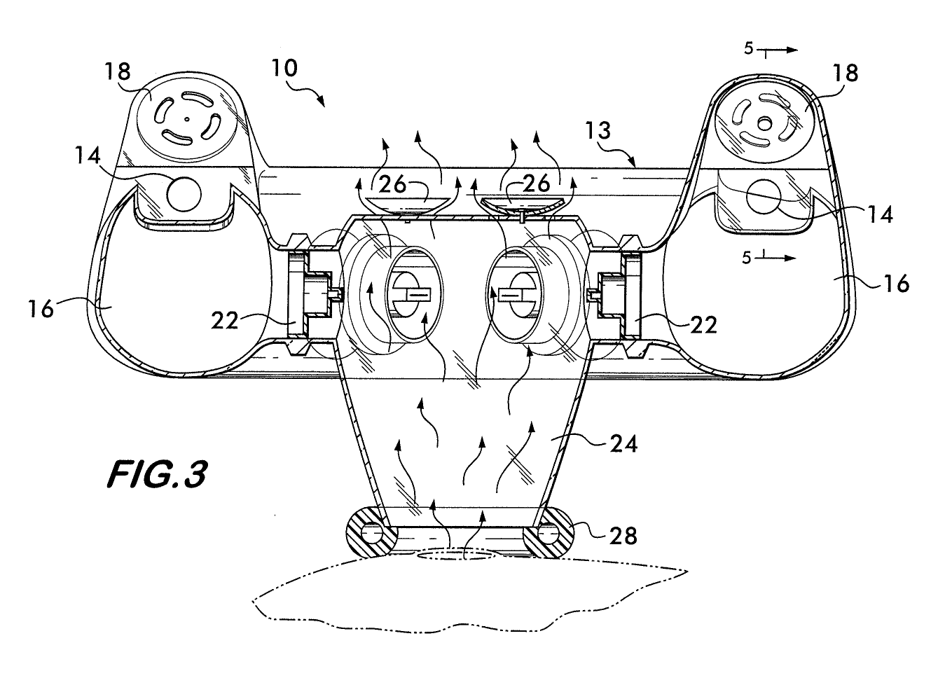

Referring now to FIG. 3, a central chamber 24 is also an integral part of the nebulizer delivery device 13. The central chamber 24 should be of the smallest possible size but large enough to allow for positioning of a sufficient quantity of exhalation one-way valves 22 such that the animal does not experience significant resistance upon exhaling. If the central chamber 24 is too large, the pressure rise during exhalation and the pressure drop during inhalation results in slower responding valves 22, 26 which increases the resistance in the system 10.

As shown in FIG. 2, a mask 28, which fits over a breathing portion of the animal, allows it to inhale the nebulized drug/air mixture from the reservoir 16 after passing through the central chamber 24. The central chamber 24 is also in communication with at least one exhalation one-way valve 26 which is shown in the closed position in FIG. 2. However, as shown in FIG. 3, when the animal exhales, the exhaled air passes through the central chamber 24, opens at least one exhalation one-way valve 26, and allows the exhaled air to escape the nebulizer delivery device 13.

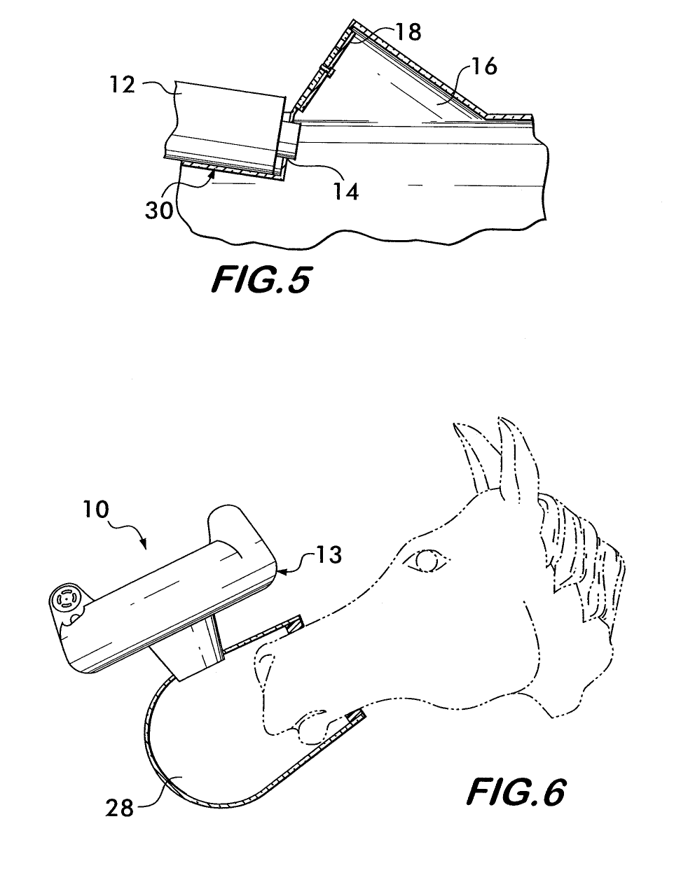

Referring now to FIGS. 4 and 5, the source 12 is, preferably, positioned in a channel 30 to prevent the source from being more easily disconnected from the nebulizing delivery device 13. Also, if desired, a nub (not shown) can be designed into the channel 30 such that, when the source 12 is connected to the delivery device 13, the source 12 is activated.

Referring now to FIGS. 2 and 4, directional arrows show a nebulized drug entering the reservoir 16 from the source 12 after passing by the connecting means 14. Directional arrows also show outside air entering the reservoir 16 through an open air inlet valve 18. As described above in more detail, the nebulized drug enters the reservoir 16 and the air inlet valve 18 opens allowing outside air to enter the reservoir 18 upon inhalation by an animal. FIG. 5 is similar to FIG. 4 and shows the source 12, connecting means 14, reservoir 16, air inlet valve 18 and channel 30. However, unlike FIGS. 2 and 4, FIGS. 3 and 5 show the air inlet valve 18 in a closed position which is its normal position when the nebulizer delivery device 13 is not in use and also when the animal exhales.

As described above and shown in FIGS. 2 and 3, the nebulizer delivery device 13 provides a pathway for the animal to exhale and a partially separate pathway for the animal to inhale. Preferably, the source 12 is capable of replenishing the reservoir 16 with more nebulized drug between exhalation/inhalation cycles. As a result of its design, nearly the entire nebulized drug constantly moves throughout the reservoir 16 until exiting the reservoir 16 through the mask 28 upon inhalation by the animal.

Referring now to FIG. 6, the versatility of the system 10 of the present invention and, in particular, the nebulizer delivery device 13, enables it to be used effectively on other animals, including a horse. Those of ordinary skill in the art will understand and appreciate that various masks 28 can be designed within the scope and spirit of the present invention to fit over at least a breathing portion of many different types of animals. As shown in FIG. 6, the mask 28 fits over the nose and mouth of the horse's head such that when the horse exhales, the exhaled breath enters the mask and passes through the nebulizer delivery device 13 as described above.

The size and shape of the mask 28 will depend on the type of animal being treated. As is the case with the other parts of the nebulizer delivery device 13, preferably, the size of the mask 28 is adjustable and is made of a lightweight, yet durable, material so that it can easily form at least a partial seal over a breathing portion of the animal. It is also preferred that the mask 28 be detachable from the nebulizing delivery device 13 so that several different types of masks 28 can be used with the same delivery device 13. Most preferably, the materials of the nebulizer delivery device 13 are strong enough to withstand occasionally being dropped from a height of several feet. As shown in FIG. 1, it is further preferred that as many parts as possible, including the mask 28, be transparent so that, for example, the nebulized drug can be observed in the reservoir 16 and the mask 28 can be properly placed over a breathing portion of the animal being treated.

The invention is also achieved by a novel method of delivering a drug via a nebulizer system to an animal. More specifically, the method includes connecting at least one source of a drug to a nebulizer delivery device having a connecting means, a reservoir, at least one air inlet valve, inhalation and exhalation one-way valves, a central chamber and a mask. The order of the several steps necessary to accomplish the method is not to be construed as a limitation; however, the claimed sequence of steps is preferred.

In order to complete the method, a source or sources of a drug or drugs must be available for use. If the source is self-contained such that it already contains the drug to be nebulized under pressure in a vile, canister or other similar object, then it is not generally necessary to prepare the drug. However, if the source is not a self-contained type, some preparation of the drug will likely be necessary. For example, it may be necessary or desirable to dissolve and/or dilute the drug in a water solution prior to connecting the source to the connecting means of the drug delivery device. Once the drug is prepared and the source is in communication with the reservoir of the delivery device, the source can be activated. This enables the drug to become nebulized and to enter the reservoir where it continues to flow until it exits the reservoir upon inhalation by the animal. After the source is activated and a working volume of the reservoir is filled with the nebulized drug, the mask portion of the delivery device is placed over a breathing portion of the animal to be treated in order to create at least a partial seal between the mask and the animal. The mask is maintained (e.g., held) in place until the animal completes at least one inhalation cycle after which the mask can be removed. If necessary or desired, one or more of the various steps for administering a drug to an animal via the described nebulizer system can be repeated. Also, if necessary or desired, the additional step of examining the animal before, during, and/or after the nebulized drug is administered to the animal can be accomplished in order to evaluate the health of the animal and/or the administration of the nebulized drug to the animal.

### EXAMPLES

The invention is now further described with respect to the following examples. The examples are intended to be illustrative and are not intended to limit the present invention in any way.

### Example 1

A bottle nosed dolphin at a theme park was diagnosed with a severe respiratory tract infection. The dolphin was removed from its pool and taken to an examination room thereby isolating the dolphin from other dolphins. Approximately 20 staff members restrained the dolphin in the examination room. A drug solution of gentamycin (250 mg/ml) was prepared by diluting it with a saline solution to a concentration of 40 mg/ml. Two jet nebulizer sources were filled with the drug solution prior to the start of the procedure. In particular, the drug reservoirs of two Omron MicroAir nebulizers were filled with 3 milliliters each of the drug solution after which the nebulizers were connected to an embodiment of the nebulizer delivery device of the present invention by sliding the filled nebulizers into designated channels on the device.

The dolphin was restrained on the examination table and a bronchoscope was inserted through the animal's blow hole so that the lungs could be observed during the procedure. The two jet nebulizers were activated by switching on the Omron nebulizers and the nebulized drug solution was observed entering the reservoir of the nebulizer device and continuing to circulate in the reservoir.

The nebulizer delivery device was placed over the dolphin's blow hole by holding the central chamber section just above a transparent mask. The trainer's wrist was placed ahead of the dolphin's blow hole and the mask was slowly lowered over the blow hole. Soon thereafter, three umbrella valves were observed opening as the dolphin exhaled allowing the exhaled air to pass through a central chamber and to escape the nebulizer device. The dolphin first inhaled approximately 40 seconds later which caused three duckbill valves to open allowing the nebulized drug to exit the reservoir, pass through the duckbill valves and travel into the central chamber. Ultimately, the medicated mist was observed entering the dolphin's blow hole after passing through the transparent mask. Using the bronchoscope, the nebulized drug was observed traveling successfully deep into the dolphin's lungs. This was repeated for three exhalation/inhalation cycles when approximately all of the drug solution in the Omron nebulizers was depleted. The dolphin received a successful dose of 750 mg/ml of gentamycin (250 mg/ml×3 ml).

### Example 2

The same dolphin as in Example 1 was treated with the same type and concentration of drug using the same nebulizer system. However, instead of being treated in an examination room, the dolphin was treated in its pool. This location for the treatment of the dolphin was preferred as it required less time and resources while also reducing stress on the dolphin. A second dolphin was also treated using the nebulizer system. The two dolphins were successfully treated in their pool with the nebulizer system on a daily basis. Remarkably, the dolphins allowed a trainer to place the mask over the dolphins' blow holes and, upon exhalation by the dolphins, the umbrellas valves were observed opening. Upon inhalation by the dolphins, the nebulized drug was observed passing through the nebulizer device and into the dolphins' lungs.

It will be appreciated by those skilled in the art that changes could be made to the embodiments, examples and methods described above without departing from the broad inventive concept of the present invention. For example, additional embodiments include using different mask designs so that different types of animals can be successfully nebulized using the nebulizer system are considered part of the present invention. Also part of the invention is a multi-part nebulizer delivery device and a single part (e.g., molded) delivery device. It is understood, therefore, that this invention is not limited to particular embodiments or methods disclosed, but it is intended to cover modifications within the spirit and scope of the present invention as recited by the appended claims.

The nebulizer system, as well as the nebulizer delivery device and method of using the device, are effective for several reasons, including some of those listed here. First, it is a means treating animals requiring drugs for chronic conditions daily or multiple times a day without the stress or extensive resources needed to treat the animals in examination rooms. Second, the lightweight construction of the nebulizer delivery device allows it to be easily used on animals without removing them from their natural environment. Third, because the nebulizer delivery device is easy to use and operate, it can be used effectively on animals with varying levels of training. Accordingly, the present invention achieves several objectives including, most importantly, successfully administering nebulized drugs to animals in need of medical treatment.

I claim:

1. A nebulizer delivery device for administering a nebulized drug to an animal comprising:
a means for connecting said device to a drug source;
a reservoir for holding said nebulized drug, wherein said reservoir is in communication with said connecting means, at least one air inlet valve and at least one inhalation one-way valve for delivering said nebulized drug to said animal;
a central chamber in communication with said reservoir and at least one exhalation one-way valve; and
a mask for receiving said nebulized drug from said reservoir and for fitting over a breathing portion of said animal.

2. The device of claim 1, wherein said drug is a water-soluble medication.

3. The device of claim 2, wherein said medication is selected from a group consisting of steroids, bronchodilators, demucelents and antibiotics.

4. The device of claim 1, wherein the volume of said reservoir is less than the respiratory system volume of said animal.

5. The device of claim 1, wherein said reservoir is shaped to promote flow of said nebulized drug in said reservoir until said drug is administered to said animal.

6. The device of claim 5, wherein said reservoir is donut-shaped.

7. The device of claim 1, wherein said connecting means is positioned to promote flow of said nebulized drug in said reservoir.

8. The device of claim 1, wherein said mask partially forms an air-tight seal between said device and said animal.

9. The device of claim 1, wherein said at least one inhalation one-way valve is a duckbill-type valve.

10. The device of claim 1, wherein said at least one air inlet valve and said at least one exhalation one-way valve are umbrella-type valves.

11. The device of claim 1, wherein said mask is transparent.

12. The device of claim 1, wherein materials of said device resist condensation of said nebulized drug.

13. The system of claim 1, wherein the volume of said central chamber is smaller than the volume of said reservoir.

14. A nebulizer system for administering a nebulized drug to an animal comprising:
at least one source of a drug for nebulization, wherein said source is connectable to a nebulizer delivery device, said device comprising:
a means for connecting said source to said device;
a reservoir for holding said drug, wherein said reservoir is in communication with said connecting means, at least one air inlet valve and at least one inhalation one-way valve for delivering said nebulized drug to said animal;
a central chamber in communication with said reservoir and at least one exhalation one-way valve; and
a mask for receiving said nebulized drug from said reservoir and for fitting over a breathing portion of said animal.

15. The system of claim 14, wherein said at least one source is a jet-type nebulizer containing said drug.

16. The system of claim 14, wherein said at least one source comprises more than one drug for administering to said animal.

17. A method of administering a drug via a nebulizer system to an animal comprising the steps of:
a) connecting at least one source of a drug to a nebulizer delivery device comprising a connecting means, a reservoir, at least one air inlet valve, inhalation and exhalation one-way valves, a central chamber and a mask;
b) activating said at least one source such that said drug is nebulized and fills said reservoir;
c) placing said mask over a breathing portion of said animal;
d) maintaining said mask over said breathing portion of said animal until said animal completes at least one exhalation/inhalation cycle,
wherein said reservoir is in communication with said connecting means, said at least one air inlet valve and said inhalation one-way valve such that inhalation by said animal administers said nebulized drug to said animal through said central chamber and said inhalation one-way valve and exhalation by said animal is exported through said central chamber and said exhalation one-way valve.

18. The method of claim 17, further comprising repeating at least one of steps (a) through (d).

19. The method of claim 17, further comprising the step of preparing said drug prior to said connecting step by dissolving a majority of said drug in a solution.

20. The method of claim 17, further comprising the step of examining said animal to evaluate said administration to said animal.
